(19) United States
(12) Patent Application Publication
(10) Pub. No.: US 2017/0319735 A1
(43) Pub. Date: Nov. 9, 2017

(54) WASTE TANK FILTER FOR A VEHICLE

(71) Applicant: Brian Keith Russell, Phoenix, AZ (US)

(72) Inventor: Brian Keith Russell, Phoenix, AZ (US)

(21) Appl. No.: 15/437,303

(22) Filed: Feb. 20, 2017

Related U.S. Application Data
(60) Provisional application No. 62/332,983, filed on May 6, 2016.

(51) Int. Cl. A61L 9/16; B01J 39/18; B01J 39/04; B63J 99/00; B60R 15/00

(52) U.S. Cl. CPC A61L 9/16; B01J 39/18; B01J 39/04; A61L 2209/14; B60R 15/00; B63J 99/00

(57) ABSTRACT

Some embodiments of the invention include a filter for a waste tank, the filter including a housing with a pair of end fittings; an inner tube positioned within the housing, the inner tube having perforated walls; and a negatively charged resin positioned between the housing and the inner tube. The first end fitting may be configured to attach to the tank's vent line, and the second end fitting may be configured to attach to external venting tubes such that fumes from the waste tank are configured to flow from the waste tank, through the inner tube of the filter, and away from the waste tank.

### CROSS-REFERENCE TO RELATED APPLICATIONS

This application claims priority to U.S. Provisional Application No. 62/332,983 filed May 6^{th}, 2016. The content of the above application is incorporated by reference in its entirety.

### FIELD OF THE DISCLOSURE

This disclosure relates generally to the field of filtration devices, and more particularly, to a filter for a waste tank in a vehicle, such as a boat or a recreational vehicle (RV).

### BACKGROUND

Holding tanks in a vehicle such as a boat or RV are populated by two different types of bacteria, aerobic and anaerobic. Both of these bacteria need oxygen to survive and breakdown the solid waste in the tanks. Aerobic bacteria break down the solid organic matter in the tank, creating as a by-product carbon dioxide, which is odorless. Once the purest oxygen in the tank has been consumed, anaerobic bacteria that can better handle the low oxygen environment takes over, and the byproduct it creates is a variety of foul smelling gasses including sulfur monoxide and sulfur dioxide.

Conventional filters for waste tanks on vehicles, such as boats and RVs, are carbon filters. These carbon filters are meant to filter the vent line air of a holding tank to remove odor while maintaining air flow in the tank. These carbon filters can become blocked over time and prevent airflow and fresh oxygen to the tank.

In order for a waste tank not to smell, the tank must breathe such that the good bacteria can survive. Thus, the blocked airflow with conventional filters causes the tanks to unnecessarily and undesirably smell. This is particularly an issue in boats, where the waste tank is contained in a compartment and the trapped odor permeates through the walls of the tanks, resulting in the boat and the surrounding areas to stink. Moreover, with conventional filters, a vacuum is created when the tank is pumped, which results in waste never being completely pumped from the tank.

More Reference to, and discussion of, the foregoing background is not presented as prior art and is respectfully submitted that none of the above-indicated disclose, teach, suggest, show, or otherwise render obvious, either singly or when considered in combination, the invention described and claimed herein.

For the foregoing reasons, there is a need for a filter that allows air to flow to and from the waste tank to promote aerobic bacterial, while simultaneously absorbing odors.

### SUMMARY

The disclosure presented herein relates to a filter for a waste tank. Some embodiments of the invention include a filter for a waste tank, the filter including a housing with a pair of end fittings; an inner tube positioned within the housing, the inner tube having perforated walls; and a negatively charged resin position between walls of the housing and the inner tube. The first end fitting may be configured to attach to the tank's vent line, and the second end fitting may be confirmed to attach to external venting tubes such that fumes from the waste tank are configured to flow from the waste tank, through the inner tube of the filter, and away from the waste tank. The preceding and following embodiments and descriptions are for illustrative purposes only and are not intended to limit the scope of this disclosure. Other aspects and advantages of this disclosure will become apparent from the following detailed description.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure. Also, the drawings included herein are considered by the applicant to be informal.

FIG. 1 is a cross sectional side view of the filter.

FIG. 2 is a side view of the filter.

FIG. 3 is a cut-away top view of the filter.

FIG. 4 is a isometric view of the filter in a waste tank system.

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

The filter of the present disclosure may be used to allow air to flow to and from the waste tank, to promote aerobic bacteria inside the tank, while simultaneously absorbing odors. The list of possible constituent elements in intended to be exemplary only, and it is not intended that the list be used to limit the device of the present application to just these elements. Persons having ordinary skill in the art relevant to the present disclosure may understand there to be equivalent elements that may be substituted with the present disclosure without changing the essential function or operation of the device.

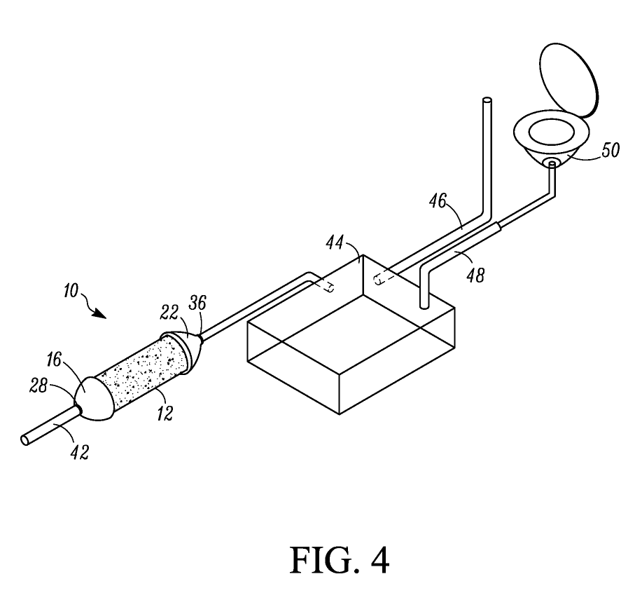

FIG. 4 shows a preferred version of the waste tank filter 10 as it could be positioned in a vehicle, such as a boat or an RV, the filter 10 comprising a housing 12 with a pair of end fittings 16 and 22, and a negatively charged resin 26. Shown at the top of end fitting 22 is end connector 36 that may be configured to attach to a vehicle's waste holding tank vent line in some embodiments. In this embodiment, oxygen would flow freely from the vehicle waste tank 44 into the filter 10 through connector 36, and out the filter through connector 28.

In this embodiment waste would travel from the toilet discharge line 48 into the waste holding tank 44. For a waste tank not to smell or leak offensive odors to the passengers of the vehicle, the tank must breathe such that the good bacteria can survive. Oxygen and the odor form the waste holding tank 44 flow through the holding tank vent line into filter 10 through connector 36. When ready to empty the waste holding tank, the waste is emptied through holding tank pump 46.

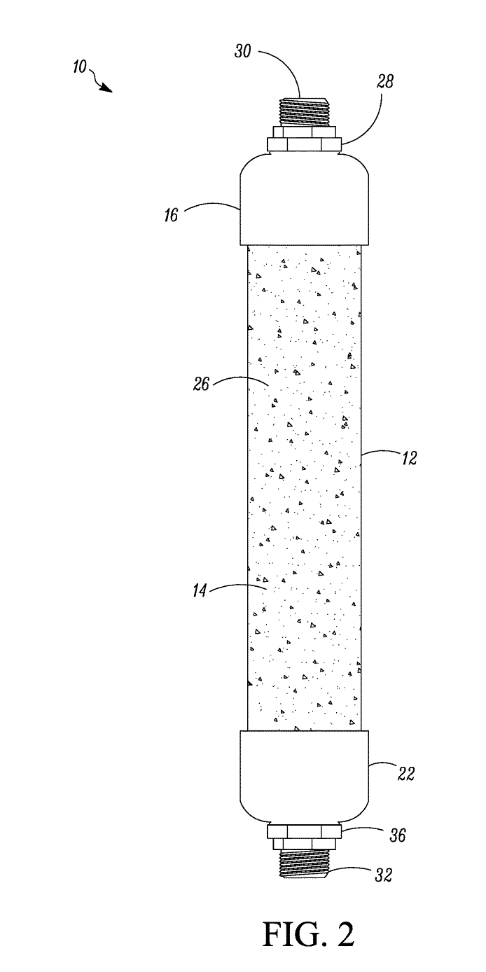

FIG. 2 shows a side view of filter 10. In this embodiment the housing 12 can be seen with two end fittings 16 and 22 and connectors 28 and 36. In this embodiment each connector is fitted with screw threads 30 and 32 to allow connection of the waste tank vent line external vent line 42.

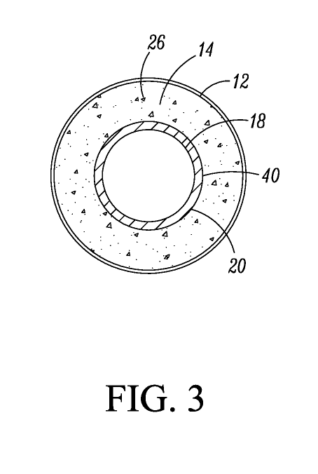

FIG. 3 Shows a cut away top down view of the filter from FIG. 2. In this embodiment, inner tube 18 is positioned within the housing 12, the inner tube 18 having perforated walls 20; and a negatively charged resin 26 positioned between the walls of the housing 12 and the inner tube 18. In some embodiments, perforated walls 20 are of a size smaller than the negatively charged resin, preventing the resin from penetrating into the inner tube but allowing oxygen and the odor to flow through the perforated tube 18.

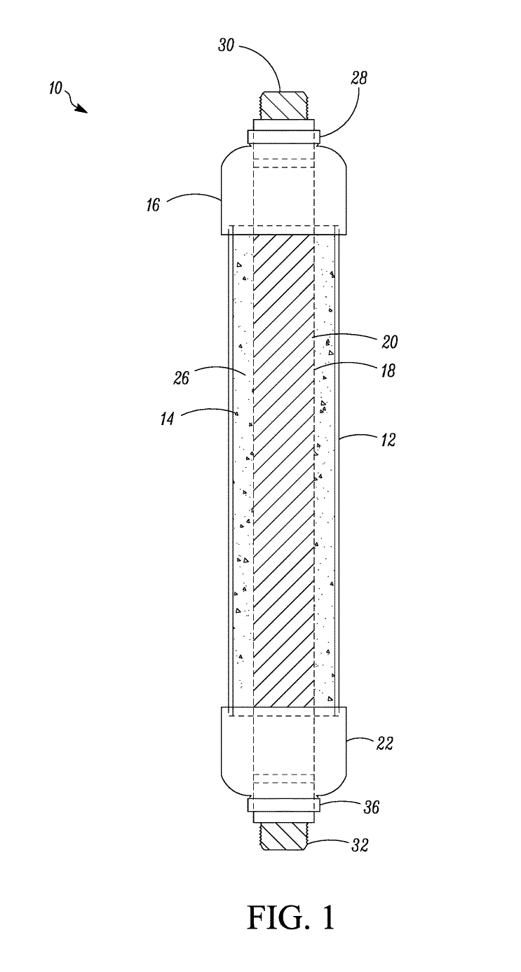

FIG. 1 shows a cross sectional side view of the filter in FIG. 2. In this embodiment, the first end fitting 22 connector 36 is configured to attach to the waste tank's vent line. Air freely flows from the tank's vent line through connector 36 and into the inner tube 18. While in the inner tube, the air from the waste tank freely flows through the inner tube 18 through the perforated walls 20, and interacts with the negatively charged resin 26 that is located in between the inner tube 18 and filter housing 12. The negative charged resin 26 attracts the odor molecules in the air flow from the waste tank to prevent the foul smell from reaching the boating occupants. Once the foul smelling odor molecules have been removed by the filter, the air freely vents from the filter through connector 28 located at the opposite end of the filter that was connected to the vent line of the waste tank.

In one embodiment, connector 28 is further connected to an external vent line to vent the air from a location away from the filter, and out of the system.

In embodiments, the negative charged resin 26 is negative charged ion pellets 14. In this embodiment, the negatively charged ion pellets 14 are larger than the perforated walls 20, preventing the pellets from getting inside inner tube 18.

In one embodiment, the second end fitting 16 through connector 28 is configured to attach to external venting tubes such fumes from the waste tank are configured to flow from the waste tank, through the inner tube 18 of the filter 10, and out the system. The filter 10 may be used in place of conventional filters for waste tanks. In embodiments, the perforated inner tube 18 may comprise or be overlapped with mesh walls 40 wherein the size of the mesh 40 is smaller than the size of the resin pellets 14 such that the resin pellets stay positioned between the inner tube 18 and the housing 12.

Any suitable negatively charged resin 26 may be used and, in some embodiments, the resin 26 comprises pellets 14 of a cation exchange resin. A negatively charged resin 26 may be used because the negatively charged pellets attract the positively charged odor, resulting in the odor being eliminated as the air flows through the inner tube 18.

The filter 10 of the present disclosure may have any desired shape and size. In some embodiments, the filter 10 is substantially cylindrically shaped.

To use the filter 10 of the present disclosure, a user may simply insert the filter 10 in place of a conventional filter. In embodiments, the filter 10 may be used to vent and filter a waste tank in, for example, a boat or an RV.

While preferred and alternate embodiments have been illustrated and described, as noted above, many changes can be made without departing from the spirit and scope of the WASTE TANK FILTER FOR A VEHICLE. Accordingly, the scope of the WASTE TANK FILTER FOR A VEHICLE is not limited by the disclosure of these preferred and alternate embodiments. Instead, the scope of the WASTE TANK FILTER FOR A VEHICLE is determined entirely by reference to the claims. Insofar as the description above and the accompanying drawings (if any) disclose any additional subject matter that is not within the scope of the claims below, the inventions are not dedicated to the public and Applicant hereby reserves the right to file one or more applications to claim such additional inventions.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example of a generic series of equivalent or similar features.

Any element in a claim that does not explicitly state “means for” performing a specified function, or “step for” performing a specific function is not to be interpreted as a “means” or “step” clause as specified in 35. U.S.C. §112 ¶6. In particular, the use of “step of” in the claims herein is not intended to invoke the provisions of U.S.C. §112 ¶6.

1. A filter for a waste tank, the filter comprising:
a housing with a first and second end fitting;
an inner tube positioned within the housing, the inner tube having perforated walls;
a negatively charged resin positioned between walls of the housing and the inner tube; and
wherein the first end fitting is configured to attach to a waste tank vent line and the second end fitting is configured to attach to external venting tubes such that fumes from the waste tank are configured to flow from the waste tank, through the inner tube of the filter, and away from the waste tank.

2. A filter as in claim 1 wherein said housing comprises:
a first and second end cap, wherein said end cap holds in place said first and second end fitting.

3. The filter as in claim 1 wherein:
said inner tube further comprises a mesh layer that overlaps said inner tube.

4. The filter as in claim 1 wherein:
said inner tube is comprised of a mesh material.

5. The filter of claim 1 wherein:
said inner tube's perforated walls are smaller than the size of said negatively charged resin.

6. The filter of claim 1 wherein:
the negatively charged resin is comprised of pellets of a cation exchange resin.

7. The filter of claim 1 wherein:
the filter housing is substantially cylindrical in shape.

8. The filter of claim 6 wherein:
said pellets are prevented from falling out of the filter by the end fittings and end caps.

9. A waste tank filter adapted to filter positively charged odorous gas from the gasses freely flowing through said filter, comprising:
a shell with a first end connector at one end and a second end connector located at the opposite end;
a perforated inner tube connected to said first and second end connector inside said shell; and
a negatively charged resin positioned between walls of said shell and said inner tube,
wherein said first end connector is configured to attach to the tank's vent line and said second end connector is configured to attach to external venting tubes such that gases from the waste tank are configured to flow from the waste tank, through the inner tube of the filter, and away from the waste tank.

10. The filter as in claim 9 wherein said shell comprises:
a first and second end cap, wherein said end cap holds in place said connectors

11. The filter as in claim 9 wherein:
said perforated inner tube further comprises a mesh layer that overlays said inner tube.

12. The filter as in claim 9 wherein:
said perforated inner tube is a mesh filter.

13. The filter of claim 9 wherein:
said perforated inner tube's walls are smaller than the size of said negatively charged resin.

14. The filter of claim 9 wherein:
the negatively charged resin is comprised of pellets of a cation exchange resin.

15. The filter of claim 9 wherein:
the filter housing is substantially cylindrical in shape.

16. The filter of claim 10 wherein:
said end caps contain said negative resin inside the filter.

17. A method of filtering air from a waste with a filter comprising:
providing a housing with a pair of end fittings configurable to attach one side of said housing to a waste tank vent line;
directing the air from the waste tank through the first end fitting and then through an inner tube positioned inside said housing, the inner tube having perforated walls;
exposing said air through said perforated walls to a negatively charged resin positioned between walls of the housing and the inner tube; and
directing said air through said second end fitting and away from the waste tank.

18. The method described in claim 17 further comprising:
directing the air through a mesh layer after exciting the inner tube with perforated walls.

19. The method described in claim 17 wherein:
said negatively charged resin is resin pellets.

20. The method described in claim 17 further comprising:
the negatively charged resin attracting the positively charged odor for the air, resulting in the odor being eliminated as the fir flows through the inner tube.
